(19) United States
(12) Patent Application Publication
(10) Pub. No.: US 2004/0072879 A1
(43) Pub. Date: Apr. 15, 2004

(54) Crystalline 2-[(2-aminoethoxy)methyl]-4-(2-chlorophenyl)-3-ethoxycarbonyl-5-methoxycarbonyl-6-methyl-1,4-dihydropyridine maleate salt (Amlodipine)

(75) Inventors: Sajja Eswaraiah, Hyderabad (IN); Ganta Madhusudan Reddy, Hyderabad (IN); Jambula Mukunda Reddy, Hyderabad, IN (US); Kammili Venkata Rambabu, Hyderabad (IN); Bolugoddu Vijaya Bhaskar, Hyderabad (IN)

Correspondence Address: LADAS & PARRY, 26 WEST 61ST STREET, NEW YORK, NY, 10023, US

(73) Assignee: DR. REDDY'S LABORATORIES LIMITED

(21) Appl. No.: 10/269,095

(22) Filed: Oct. 10, 2002

(51) Int. Cl. C07D 211/82; A61K 31/455

(52) U.S. Cl. 514/355; 546/315

(57) ABSTRACT

The present invention relates to novel crystalline forms of Amlodipine Maleate These crystalline forms are useful as pharmaceutical agents. This invention also relates to pharmaceutical compositions which include these crystalline forms and to methods of treatment using these crystalline forms. The novel crystalline compounds of the present invention are useful as calcium channel blockers and are thus useful as anti-ischaemic and anti-hypertensive agents.

### FIELD OF THE INVENTION

The present invention relates to novel crystalline forms of Amlodipine Maleate which is known by the chemical name 2-[(2-aminoethoxy)methyl]4-(2-chlorophenyl)-3-ethoxycarbonyl-5-methoxycarbonyl-6-methyl-1,4-dihydropyridine maleate salt. These crystalline forms are useful as pharmaceutical agents. This invention also relates to pharmaceutical compositions which include these crystalline forms and to methods of treatment using these crystalline forms. The novel crystalline compounds of the present invention are useful as calcium channel blockers and are thus useful as anti-ischaemic and anti-hypertensive agents.

### BACKGROUND OF THE INVENTION

U.S. Pat. No. 4,572,909, which is incorporated by reference herein discloses a class of substituted dihydropyridine derivatives which are described as being useful calcium channel blockers. One of the most preferred compounds identified in this patent is 2-[(2-aminoethoxy)methyl]-4-(2-chlorophenyl)-3-ethoxycarbonyl-5-methoxycarbonyl-6-methyl-1,4-dihydropyridine, having the generic name, Amlodipine.

U.S. Pat. No. 4,572,909, discloses several different pharmaceutically acceptable acid addition salts of Amlodipine. In particular, the pharmaceutically acceptable acid addition salts are said to be those formed from acids which form non toxic acid anions such as chloride, bromide, sulphate, phosphate, acetate, maleate, lactate, tartrate, citrate, gluconate etc. of these salts maleate is disclosed as being particularly preferred. It exists in various salt forms amongst which is Amlodipine Besylate. Amlodipine Besylate is commercially available as Norvasc in the form of oral tablets by Pfizer in 2.5 mg, 5 mg and 10 mg base preparations. Therapeutically Amlodipine belongs to the class of antianginals and antihypertensives. The main mechanism of action of Amlodipine is the inhibition of calcium channels. It is also available in combination with diuretics and angiotensin converting enzyme inhibitors.

Use of Amlodipine in the therapy of cardiovascular disorders is known. Patent specification AU1354000 discloses a method for treating hypertension, angina and other disorders using optically pure (−) Amlodipine. U.S. Pat. No. 6,080,761 discloses the inhibition of smooth muscle migration by (R) Amlodipine. Flynn J T et al. describes the Treatment of hypertensive children with Amlodipine in Am. J. Hypertens., (AJHYE6, 08957061); 2000; Vol. 13 (10); pp. 1061-1066. Marche P discloses Amlodipine and the mechanisms of vascular hypertrophy in Drugs (DRUGAY, 00126667); 2000; Vol.59 (Spec. Issue 2); pp. 1-7. Burges R A explains the Pharmacologic profile of Amlodipine Am. J. Cardiol. (AJCDAG, 00029149); 1989; Vol.64 (17); pp. 101-201.

U.S. Patent Application Publication 2002/0086888 of Jul. 4, 2002 and WO 02/053542 describe a number of processes for preparing amlodipine maleate.

Example 1 of United States Patent Application Publication US 2002/0086888 describes the preparation of Amlodipine Maleate. The differential scanning calorimetry thermogram of this compound is shown in FIG. 2 of United States Patent Application Publication US 2002/0086888.

The X-Ray diffraction pattern of this compound is shown in FIG. 3 of United States Patent Application Publication US 2002/0086888.

The ability of a substance to exist in more than one crystal form is defined as polymorphism and these different crystal forms are named “polymorph modifications” or “polymorphs”. In general, polymorphism is affected by the ability of a molecule of a substance to change its conformation or to form different intermolecular or intra-molecular interactions, particularly hydrogen bonds, which is reflected in different atom arrangements in the crystal lattices of different polymorphs. Polymorphism is found in several organic compounds.

The different polymorphs of a substance possess different energies of the crystal lattice and, thus, in solid state they show different physical properties such as form, density, melting point, colour, stability, dissolution rate, milling facility, granulation, compacting, etc., which in medicaments may affect the preparation of pharmaceutical forms, their stability, dissolution and bioavailability and, consequently, their action.

J. Haleblian, W. McCrone, J. Pharm. Sci. 58 (1969) 911; L. Borka, Pharm. Acta Helv. 66 (1991) 16; M. Kuhnert-Brandstatter, Pharmazie 51 (1996) 443; H. G. Brittain, J. Pharm. Sci. 86 (1997) 405; W. H. Streng, DDT 2 (1997) 415; and K. Yoshii, Chem. Pharm. Bull. 45 discuss polymorphism of compounds. A good knowledge of polymorphism is useful in the process of medicament development.

There is hence a need to produce Amlodipine maleate in pure and crystalline form to enable formulations to meet exacting pharmaceutical requirements and specifications.

### SUMMARY OF THE INVENTION

It has now surprisingly been found that the substance Amlodipine Maleate can exist in different crystalline forms and the novel crystalline polymorphic forms of Amlodipine maleate described herein are designated as Form I and Form II. They are characterized by their X-ray diffraction (XRD) pattern and differential scanning calorimetry (DSC).

Methods for the preparation of the polymorphic forms I, II and III of Amlodipine maleate are also described herein.

Pharmaceutical formulations containing the novel polymorphic forms of Amlodipine maleate are described herein.

The use of the polymorphic forms to treat cardiovascular conditions is also described.

### BRIEF DESCRIPTION OF THE FIGURES

FIG. 1 is characteristic X-ray powder diffractogram of Form I of Amlodipine Maleate of this invention.

FIG. 2 is Differential Scanning Calorimetry thermogram of Form I of Amlodipine Maleate of this invention.

FIG. 3 is characteristic X-ray powder diffractogram of Form II of Amlodipine Maleate of this invention.

FIG. 4 is Differential Scanning Calorimetry thermogram of Form II of Amlodipine Maleate of this invention.

### DETAILED DESCRIPTION OF THE INVENTION

Novel crystalline Form I and Form II of Amlodipine Maleate may be characterized by their X-Ray powder diffraction patterns and Differential Scanning Calorimetry thermograms. The X-Ray diffraction patterns of Form I and Form II of Amlodipine Maleate were measured on a Bruker Axs, D8 Advance Powder X-ray Diffractometer with Cu K alpha-1 Radiation source and DSC were recorded on Perkin Elmer Pyris-6 DSC with a N₂ flow of 30 mL/min.

Crystalline Form I has X-ray powder diffraction pattern essentially as shown in Table 1. The X-ray powder diffraction pattern is express in terms of the 2θ values and relative intensities (cps).

TABLE 1

| | Intensity |
|---|---|
| 2θ (°) | (cps) |
| 4.459 | 41.1 |
| 8.871 | 34.3 |
| 10.593 | 3.0 |
| 11.160 | 1.7 |
| 11.488 | 12.0 |
| 12.700 | 9.1 |
| 13.296 | 100.0 |
| 13.590 | 41.2 |
| 15.694 | 36.2 |
| 17.730 | 10.3 |
| 18.156 | 42.1 |
| 18.608 | 4.5 |
| 19.208 | 33.3 |
| 19.772 | 4.8 |
| 20.126 | 10.6 |
| 20.908 | 7.0 |
| 21.523 | 29.9 |
| 21.964 | 95.6 |
| 22.985 | 21.3 |
| 23.687 | 29.4 |
| 24.584 | 27.1 |
| 25.085 | 4.9 |
| 25.538 | 4.1 |
| 26.269 | 27.2 |
| 26.753 | 13.2 |
| 27.504 | 17.8 |
| 28.095 | 11.5 |
| 29.972 | 9.0 |
| 30.433 | 2.9 |
| 31.175 | 12.4 |
| 31.824 | 8.0 |
| 32.671 | 2.9 |
| 33.564 | 7.7 |
| 34.132 | 12.6 |
| 36.223 | 6.1 |
| 37.836 | 8.4 |
| 38.776 | 4.2 |
| 39.914 | 5.7 |

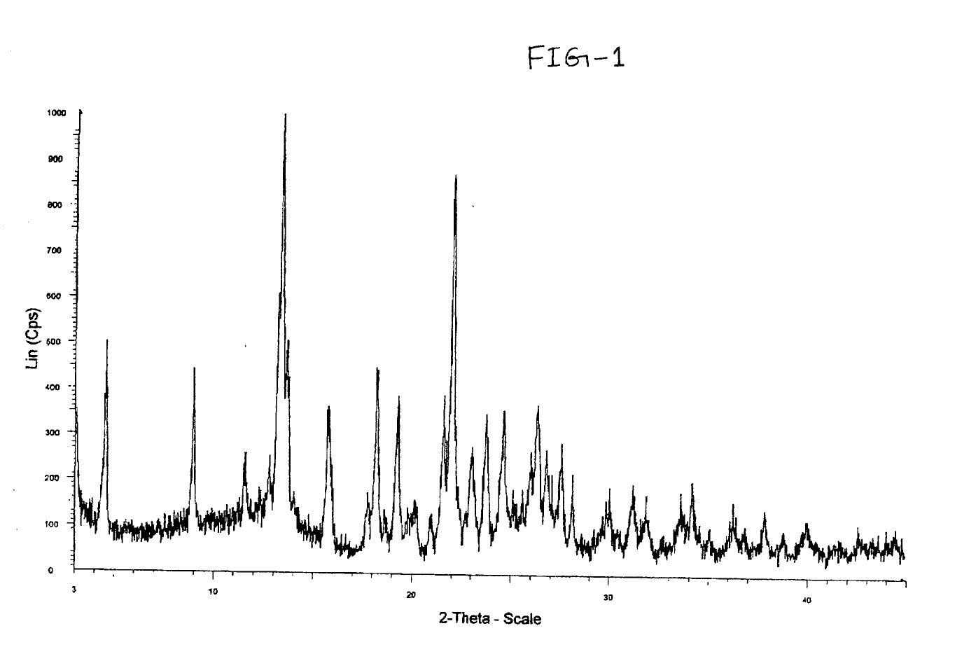

The present invention thus provides Form I of Amlodipine Maleate that is characterized by its X Ray powder diffraction, substantially in accordance with FIG. 1.

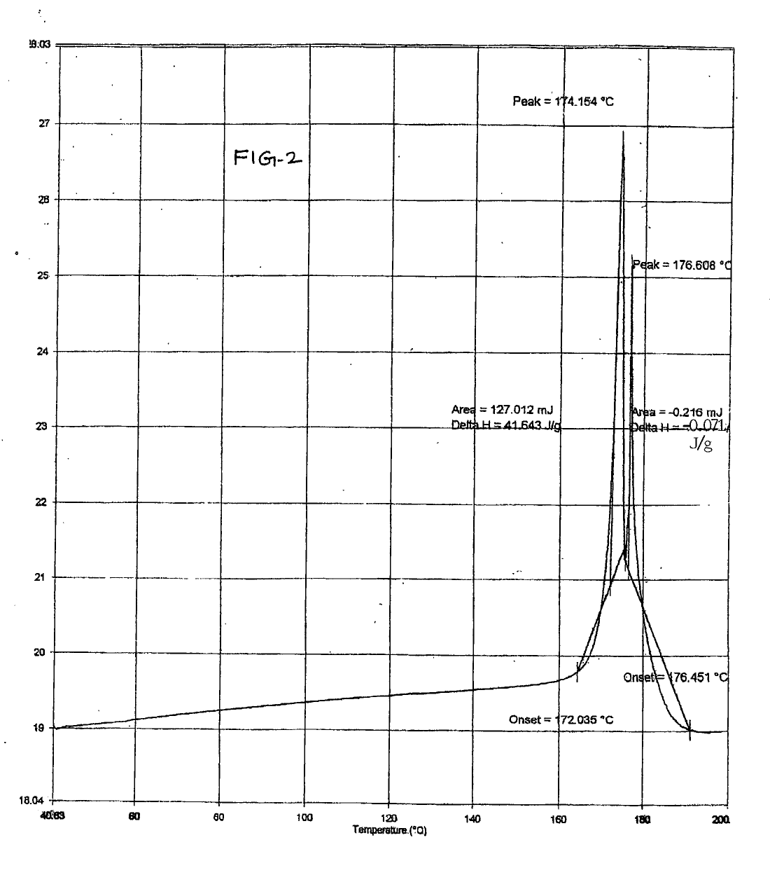

Form I of Amlodipine maleate is further characterized by the Differential Scanning Calorimetry thermogram of Form I of Amlodipine Maleate. The Differential Scanning Calorimetry thermogram at the heating rate of 5° C./min exhibits endo-endo peaks at 174.1 and 176.6° C. substantially in accordance with FIG. 2.

Another aspect of the invention provides, a process for the preparation of Form I of Amlodipine Maleate, which comprises the steps of:

a. dissolving maleic acid in a solvent or a mixture of solvents, followed by addition of Amlodipine base;

b. maintaining the reaction mixture of step a at a temperature ranging from ambient to the reflux temperature of the solvent used; and

c. isolating Form I of Amlodipine Maleate thus obtained.

Form I of Amlidipine Maleate may be isolated by methods known in the art.

The single solvent used in step a is selected from C₂-C₆ straight or branched esters selected from methyl acetate, ethyl acetate, n-butyl acetate, tertiary butylacetate or methyl tertiary butyl acetate, preferably tertiary butylacetate. The mixture of solvents used in step a. are selected from “n-butanol and methyl tertiary butyl ether”, “toluene and isopropyl alcohol”, “tertiary butanol and cyclohexane or tetrahydrofuran” or “n-butanol dioxane, n-propylacetate, toluene, diisopropylether, or cyclohexane and tertiary butyl acetate”. Whenever a mixture of solvents is used for preparation of Form-I, the ratio of more polar solvent to the less polar solvent used in the mixture is 1:1-10 v/v; preferably 1:1; more preferably 1:5 v/v and most preferably 1:10 v/v.

Crystalline Form II has X-ray powder diffraction pattern essentially as shown in the Table 2. The X-ray powder diffraction pattern is expressed in terms of the 2θ values, and relative intensities (cps).

TABLE 2

| | Intensity |
|---|---|
| 2θ (°) | (cps) |
| 4.421 | 54.1 |
| 8.851 | 29.5 |
| 10.147 | 2.7 |
| 11.421 | 14.7 |
| 12.032 | 4.8 |
| 12.644 | 12.5 |
| 13.071 | 62.5 |
| 13.297 | 73.5 |
| 13.490 | 100 |
| 14.435 | 4.8 |
| 14.838 | 7.1 |
| 15.655 | 40.2 |
| 17.052 | 7.0 |
| 17.561 | 12.7 |
| 18.072 | 28.4 |
| 18.611 | 21.0 |
| 19.186 | 19.9 |
| 19.692 | 15.2 |
| 20.078 | 9.6 |
| 21.237 | 25.2 |
| 21.958 | 85.2 |
| 22.912 | 19.0 |
| 23.536 | 23.3 |
| 24.191 | 15.3 |
| 24.493 | 16.2 |
| 25.058 | 17.2 |
| 25.932 | 11.1 |
| 26.264 | 29.1 |
| 26.717 | 13.4 |
| 27.250 | 28.5 |
| 28.051 | 5.3 |
| 29.778 | 11.3 |
| 31.728 | 5.0 |
| 33.512 | 10.7 |
| 33.987 | 9.7 |
| 35.233 | 1.5 |
| 37.085 | 3.8 |
| 40.536 | 3.7 |

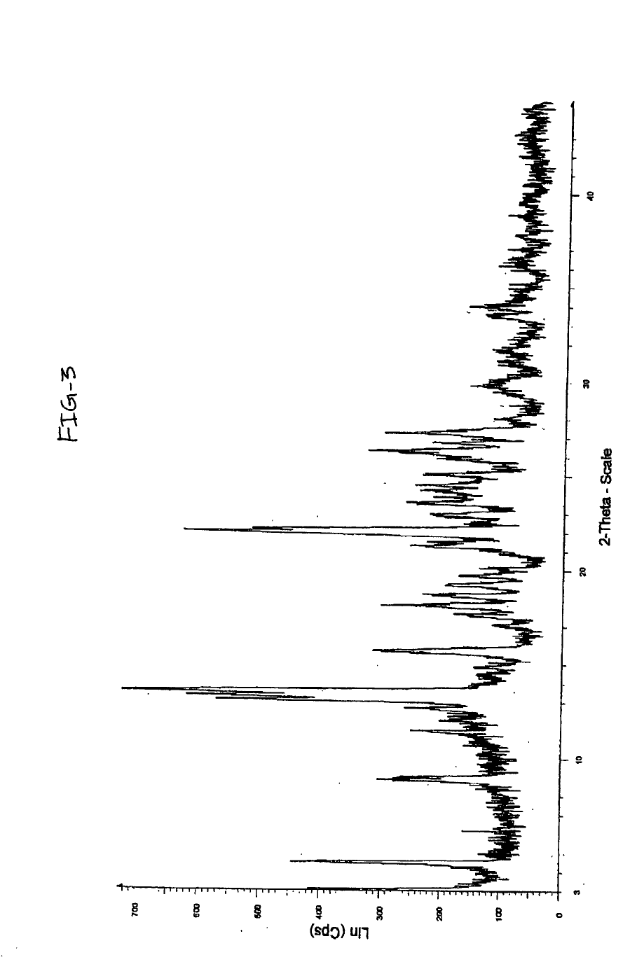

The present invention thus provides Form II of Amlodipine Maleate that is characterized by its X Ray powder diffraction substantially in accordance with FIG. 3.

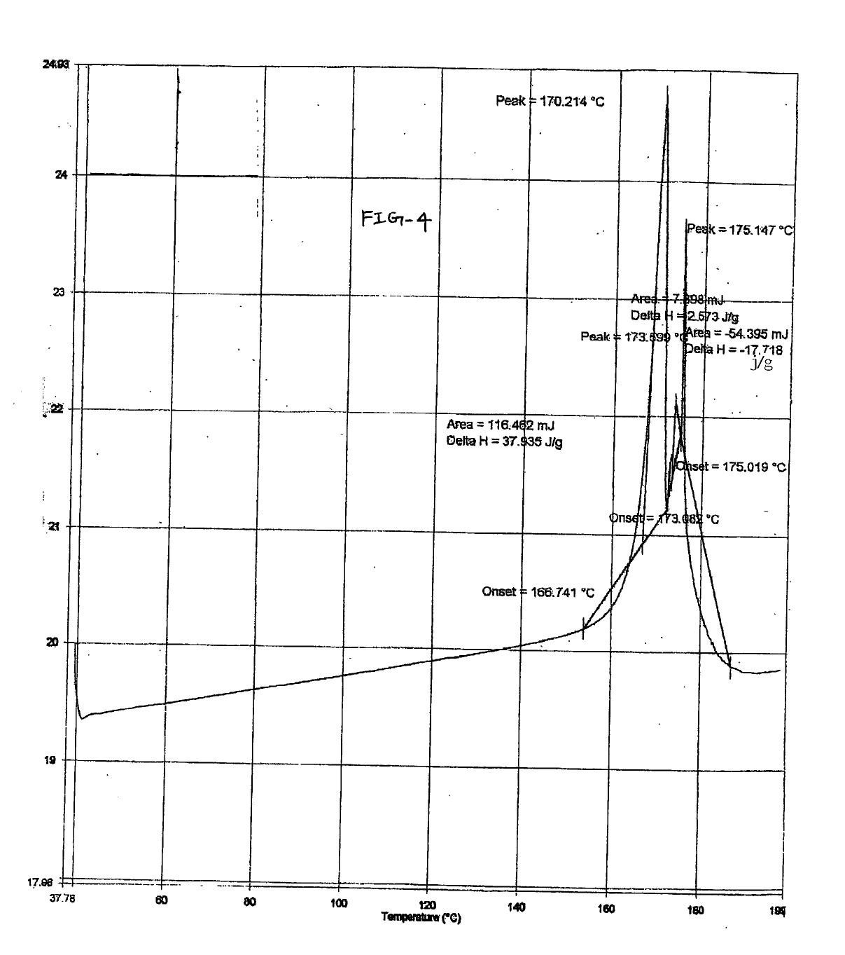

Form II of Amlodipine maleate is further characterized by the Differential Scanning Calorimetry thermogram of Form II of Amlodipine Maleate. The Differential Scanning Calorimetry thermogram exhibits endo-endo peaks at a heating rate of 5° C./min about 170.2, 173.6 and 175.1° C. substantially in accordance with FIG. 4.

Another aspect of the present invention provides a process for the preparation of Form II of Amlodipine Maleate, which comprises the steps of:

i. dissolving maleic acid in a mixture of solvents followed by addition of Amlodipine base;

ii. maintaining the reaction mixture of step i., at a temperature ranging from ambient to the reflux temperature of the solvent; and

iii. isolating Form II of Amlodipine Maleate thus obtained.

Form II of Amlidipine Maleate can be isolated by methods known in the art. The mixture of solvents used in step i are selected from “n-propanol and diethylether”, “ethyl acetate and toluene”, “methyl isobutyl ketone and tertiary butyl acetate” and “tertiary butanol and diisopropylether”. The ratio of more polar solvent to the less polar solvent used in the mixture is is 1:1-10 v/v, preferably 1:1 v/v, more preferably 1:5 v/v and most preferably 1:10 v/v.

The Amlodipine base used in the process for preparation of novel Forms I and II of Amlodipine Maleate may be prepared as per the process disclosed in U.S. Pat. No. 4,572,909. The Amlodipine base may be prepared by any other known process.

The crystalline forms of Amlodipine Maleate of the present invention are produced in good yields (75-85%) and are high melting solids which are very well suited for formulation.

Crystalline Form I and Form II of Amlodipine Maleate of the present invention may exist in unsolvated as well as solvated forms. In general, both unsolvated as well as solvated forms are intended to be encompassed within the scope of the present invention.

The present invention also provides for pharmaceutical compositions containing polymorphic forms of Amlodipine maleate. The active ingredient in the composition will be administered in an amount effective to treat at least one or more cardiovascular disorders. Amlodipine maleate of this invention can be used to treat or prevent ischaemia, hypertension or can be used as a calcium channel blocker. The amount of amlodipine (as free base) administered orally will generally be 1-40 mg daily.

The oral formulations of this invention may be in the form of tablets, caplets and capsules. Other solid oral formulation are also within the scope of the invention.

The composition can be formulated into tablet dosage form using suitable commonly used diluent(s), disintegrant(s), binder(s) and lubricant(s). The disintegrant(s) used may be incorporated either intragranularly or extragranularly or partly intragranularly and partly extragranularly.

The composition can also be formulated into a bilayered tablet dosage form using suitable commonly used diluents or into a capsule dosage form.

All of the above described pharmaceutical compositions may optionally contain one or more of each of the following excipients: carriers, diluents, colorants, flavoring agents, lubricants, solubilizing agents, disintegrants, binders and preservatives.

All of the pharmaceutical compositions described above can be made by known methods and techniques. For example, the tablets can be made by dry granulation/direct compression or by a classical wet granulation method. Typically, tablets are made by blending, filling and compressing into tablets. The blending step may comprise a wet granulation or dry granulation. Similarly, capsules can be made by blending the ingredients and filling the capsule.

The present invention is illustrated by the following examples, which do not construe to limit the effective scope of the claims.

### EXAMPLES

### Example 1

Maleic acid (2.8 g) is dissolved in n-butanol (10 ml). To the clear solution methyl tertiary butyl ether (100 ml) is added at 20-30° C. and stirred for about ½ an hour till a clear solution is obtained. Amlodipine base (10 g) is then added to this reaction mixture at 20-25° C. and the reaction mixture is stirred for about 1 hour 15 minutes. The solid that precipitates out is filtered, washed with methyl tertiary butyl ether (50 ml) and then dried at 50-55° C. to yield Form I Amlodipine maleate.

Yield: 78%

### Example 2

Maleic acid is dissolved in n-butanol (10 ml) to get a clear solution. To this, methyl tertiary butyl ether (100 ml) is added at 20-30° C. and stirred for about ½ an hour till a clear solution is obtained. Amlodipine base (10 g) is then added to this reaction mixture at 20-25° C. and the reaction mixture is stirred for about 1 hour 45 minutes. The solid that precipitates out is filtered, washed with methyl tertiary butyl ether (50 ml) and then dried at 50-55° C. to yield Form I Amlodipine maleate.

Yield: 85%

### Example 3

Maleic acid is dissolved in tertiary butylacetate (120 ml) at 65-70° C. temperature. Amlodipine base (10 g) is then added to this reaction mixture at 65-70° C. and the reaction mixture is stirred for about 1 hour. The reaction mixture is then cooled to 50-55° C. and maintained at this temperature for about an hour. The solid that precipitates out is filtered, washed with tertiary butyl acetate (20 ml) and then dried at 45-50° C. to yield Form I Amlodipine maleate.

Yield: 78%

### Example 4

Maleic acid (2.8 g) is dissolved in n-propanol (10 ml). To this clear solution diethyl ether (100 ml) is added at 25-30° C. and stirred for about ½ an hour till a clear solution is obtained. Amlodipine base (10 g) is then added to this reaction mixture at 20-25° C. and the reaction mixture is stirred for about 1-2 hours. The solid that precipitates out is filtered, washed with methyl tertiary butyl ether (50 ml) and then dried at 50-55° C. to yield Form II Amlodipine maleate.

Yield: 75%

1. Novel Crystalline Form I of Amlodipine Maleate.

2. Form I of Amlodipine maleate characterized by an X-ray powder diffraction pattern with 2θ values at about 4.459, 8.871, 10.593, 11.160, 11.488, 12.700, 13.296, 13.590, 15.694, 17.730, 18.156, 18.608, 19.208, 19.772, 20.126, 20.908, 21.523, 21.964, 22.985, 23.687, 24.584, 25.085, 25.538, 26.269, 26.753, 27.504, 28.095, 29.972, 30.433, 31.175, 31.824, 32.671, 33.564, 34.132, 36.223, 37.836, 38.776, 39.914.

3. Form I of Amlodipine maleate according to claim 1, characterized by XRD pattern substantially in accordance with FIG. 1.

4. Form I of Amlodipine maleate according to claim 2, characterized by XRD pattern substantially in accordance with FIG. 1.

5. Form I of Amlodipine maleate according to claim 1, characterized by DSC pattern having endo-endo peaks at about 174.1 and 176.6° C.

6. Form I of Amlodipine maleate according to claim 2, characterized by DSC pattern having endo-endo peaks at about 174.1 and 176.6° C.

7. Form I of Amlodipine maleate according to claim 4 characterized by DSC pattern substantially in accordance with FIG. 2.

8. Novel Crystalline Form II of Amlodipine Maleate.

9. Form II of Amlodipine maleate characterized by an X-ray powder diffraction pattern with 2θ values at about 4.421, 8.851, 10.147, 11.421, 12.032, 12.644, 13.071, 13.297, 13.490, 14.435, 14.838, 15.655, 17.052, 17.561, 18.072, 18.611, 19.186, 19.692, 20.078, 21.237, 21.958, 22.912, 23.536, 24.191, 24.493, 25.058, 25.932, 26.264, 26.717, 27.250, 28.051, 29.778, 31.728, 33.512, 33.987, 35.233, 37.085, 40.536.

10. Form II of Amlodipine maleate according to claim 8, characterized by XRD pattern substantially in accordance with FIG. 3.

11. Form II of Amlodipine maleate according to claim 9, characterized by XRD pattern substantially in accordance with FIG. 3.

12. Form II of Amlodipine maleate according to claim 8, characterized by DSC pattern having endo-endo peaks at about 170.2, 173.6 and 175.1° C.

13. Form II of Amlodipine maleate according to claim 9, characterized by DSC pattern having endo-endo peaks at about 170.2, 173.6 and 175.1° C.

14. Form II of Amlodipine maleate according to claim 13, characterized by DSC pattern substantially in accordance with FIG. 4.

15. A process for the preparation of Form I of Amlodipine maleate, which comprises the steps of:
a) dissolving maleic acid in a solvent or a mixture of solvents followed by addition of Amlodipine base;
b) maintaining the reaction mixture of step a), at a temperature ranging from ambient to the reflux temperature of the solvent; and
c) isolating the Form I of Amlodipine Maleate thus obtained.

16. The process according to claim 16, wherein the solvent used in step a) is selected from C₂-C₆ straight or branched ester.

17. The process according to claim 15 or 16, wherein the C₂-C₆ straight or branched ester is selected from the group consisting of ethyl acetate, tertiary butyl acetate, methyl acetate, methyl tertiary butyl acetate and n-butyl acetate.

18. The process according to claim 15, 16 or 17, wherein the single solvent used is tertiary butyl acetate.

19. The process according to claim 15, wherein the mixture of solvents used in step a) is selected from the group consisting of n-butanol and methyl tertiary butyl ether; toluene and isopropyl alcohol; tertiary butanol and cyclohexane or tetrahydrofuran; and n-butanol, dioxane, n-propylacetate, toluene, diusopropylether, or cyclohexane and tertiary butyl acetate.

20. The process according to claims 15 or 19, wherein the mixture of solvents used is n-butanol and methyl tertiary butyl ether.

21. The process according to claim 15, 19 or 20, wherein the ratio of a more polar solvent to a less polar solvent used in the mixture of solvents is 1:1 to 10 v/v.

22. The process according to claim 21, wherein the ratio of more polar solvent to the less polar solvent used in the mixture of solvents is 1:1 to 5 v/v.

23. The process according to claim 21, wherein the ratio of more polar solvent to the less polar solvent used in the mixture of solvents is 1:1 v/v.

24. A process for the preparation of Form II of Amlodipine maleate, which comprises the steps of:
a) dissolving maleic acid in a mixture of solvents followed by addition of Amlodipine base;
b) maintaining the reaction mixture of step a) at a temperature ranging from ambient to the reflux temperature of the solvent; and
c) isolating Form II of Amlodipine Maleate thus obtained.

25. The process according to claim 24, wherein mixture of solvents used in step a), is selected from the group consisting of: n-propanol and diethylether; ethyl acetate and toluene; methyl isobutyl ketone and tertiary butyl acetate; and tertiary butanol and diisopropylether.

26. The process according to claim 25, wherein the mixture of solvents used is n-propanol and diethylether.

27. The process according to claim 24, 25 or 26, wherein the ratio of a more polar solvent to the less polar solvent used in the mixture of solvents is 1:1 to 10 v/v.

28. The process according to claim 27, wherein the ratio of a more polar solvent to the less polar solvent used in the mixture of solvents is 1:1 to 5 v/v.

29. The process according to claim 27, wherein the ratio of more polar solvent to the less polar solvent used in the mixture of solvents is 1:1 v/v.

30. A pharmaceutical formulation comprising as an active ingredient, Form I of Amlodipine maleate and a pharmaceutically acceptable carrier, excipient or diluent; wherein said Form I of Amlodipine maleate is characterized by an XRD pattern with 2θ values at about 4.459, 8.871, 10.593, 11.160, 11.488, 12.700, 13.296, 13.590, 15.694, 17.730, 18.156, 18.608, 19.208, 19.772, 20.126, 20.908, 21.523, 21.964, 22.985, 23.687, 24.584, 25.085, 25.538, 26.269, 26.753, 27.504, 28.095, 29.972, 30.433, 31.175, 31.824, 32.671, 33.564, 34.132, 36.223, 37.836, 38.776, 39.914.

31. A pharmaceutical formulation comprising as an active ingredient, Form II of Amlodipine maleate and a pharmaceutically acceptable carrier, excipient or diluent; wherein said Form II of Amlodipine maleate is characterized by an X-ray powder diffraction pattern with 2θ values at about 4.421, 8.851, 10.147, 11.421, 12.032, 12.644, 13.071, 13.297, 13.490, 14.435, 14.838, 15.655, 17.052, 17.561, 18.072, 18.611, 19.186, 19.692, 20.078, 21.237, 21.958, 22.912, 23.536, 24.191, 24.493, 25.058, 25.932, 26.264, 26.717, 27.250, 28.051, 29.778, 31.728, 33.512, 33.987, 35.233, 37.085, 40.536.

32. A pharmaceutical formulation comprising Form I of Amlodipine maleate, Form II of Amlodipine maleate and a pharmaceutically acceptable carrier, excipient or diluent; wherein said Form I of Amlodipine maleate is characterized by an XRD pattern with 2θ values at about 4.459, 8.871, 10.593, 11.160, 11.488, 12.700, 13.296, 13.590, 15.694, 17.730, 18.156, 18.608, 19.208, 19.772, 20.126, 20.908, 21.523, 21.964, 22.985, 23.687, 24.584, 25.085, 25.538, 26.269, 26.753, 27.504, 28.095, 29.972, 30.433, 31.175, 31.824, 32.671, 33.564, 34.132, 36.223, 37.836, 38.776, 39.914 and said Form II of Amlodipine maleate is characterized by the X-ray powder diffraction pattern with 2θ values at about 4.421, 8.851, 10.147, 11.421, 12.032, 12.644, 13.071, 13.297, 13.490, 14.435, 14.838, 15.655, 17.052, 17.561, 18.072, 18.611, 19.186, 19.692, 20.078, 21.237, 21.958, 22.912, 23.536, 24.191, 24.493, 25.058, 25.932, 26.264, 26.717, 27.250, 28.051, 29.778, 31.728, 33.512, 33.987, 35.233, 37.085, 40.536.

33. A method of treating ischaemia comprising administering an effective amount of Form I of Amlodipine maleate to a patient in need thereof; wherein said Form I of Amlodipine maleate is characterized by an XRD pattern with 2θ values at about 4.459, 8.871, 10.593, 11.160, 11.488, 12.700, 13.296, 13.590, 15.694, 17.730, 18.156, 18.608, 19.208, 19.772, 20.126, 20.908, 21.523, 21.964, 22.985, 23.687, 24.584, 25.085, 25.538, 26.269, 26.753, 27.504, 28.095, 29.972, 30.433, 31.175, 31.824, 32.671, 33.564, 34.132, 36.223, 37.836, 38.776, 39.914.

34. A method of treating ischaemia comprising administering an effective amount of Form II of Amlodipine maleate to a patient in need thereof; wherein said Form II of Amlodipine maleate is characterized by an X-ray powder diffraction pattern with 2θ values at about 4.421, 8.851, 10.147, 11.421, 12.032, 12.644, 13.071, 13.297, 13.490, 14.435, 14.838, 15.655, 17.052, 17.561, 18.072, 18.611, 19.186, 19.692, 20.078, 21.237, 21.958, 22.912, 23.536, 24.191, 24.493, 25.058, 25.932, 26.264, 26.717, 27.250, 28.051, 29.778, 31.728, 33.512, 33.987, 35.233, 37.085, 40.536.

35. A method of treating hypertension comprising administering an effective amount of Form I of Amlodipine maleate to a patient in need thereof; wherein said Form I of Amlodipine maleate is characterized by an XRD pattern with 2θ values at about 4.459, 8.871, 10.593, 11.160, 11.488, 12.700, 13.296, 13.590, 15.694, 17.730, 18.156, 18.608, 19.208, 19.772, 20.126, 20.908, 21.523, 21.964, 22.985, 23.687, 24.584, 25.085, 25.538, 26.269, 26.753, 27.504, 28.095, 29.972, 30.433, 31.175, 31.824, 32.671, 33.564, 34.132, 36.223, 37.836, 38.776, 39.914.

36. A method of treating hypertension comprising administering an effective amount of Form II of Amlodipine maleate to a patient in need thereof; wherein said Form II of Amlodipine maleate is characterized by an X-ray powder diffraction pattern with 2θ values at about 4.421, 8.851, 10.147, 11.421, 12.032, 12.644, 13.071, 13.297, 13.490, 14.435, 14.838, 15.655, 17.052, 17.561, 18.072, 18.611, 19.186, 19.692, 20.078, 21.237, 21.958, 22.912, 23.536, 24.191, 24.493, 25.058, 25.932, 26.264, 26.717, 27.250, 28.051, 29.778, 31.728, 33.512, 33.987, 35.233, 37.085, 40.536.

37. A method of preventing ischaemia comprising administering an effective amount of Form I of Amlodipine maleate to a patient in need thereof; wherein said Form I of Amlodipine maleate is characterized by an X-ray powder diffraction pattern with 2θ values at about 4.459, 8.871, 10.593, 11.160, 11.488, 12.700, 13.296, 13.590, 15.694, 17.730, 18.156, 18.608, 19.208, 19.772, 20.126, 20.908, 21.523, 21.964, 22.985, 23.687, 24.584, 25.085, 25.538, 26.269, 26.753, 27.504, 28.095, 29.972, 30.433, 31.175, 31.824, 32.671, 33.564, 34.132, 36.223, 37.836, 38.776, 39.914.

38. A method of preventing ischaemia comprising administering an effective amount of Form II of Amlodipine maleate to a patient in need thereof; wherein said Form II of Amlodipine maleate is characterized by an X-ray powder diffraction pattern with 2θ values at about 4.421, 8.851, 10.147, 11.421, 12.032, 12.644, 13.071, 13.297, 13.490, 14.435, 14.838, 15.655, 17.052, 17.561, 18.072, 18.611, 19.186, 19.692, 20.078, 21.237, 21.958, 22.912, 23.536, 24.191, 24.493, 25.058, 25.932, 26.264, 26.717, 27.250, 28.051, 29.778, 31.728, 33.512, 33.987, 35.233, 37.085, 40.536.

39. A method of preventing hypertension comprising administering an effective amount of Form I of Amlodipine maleate to a patient in need thereof; wherein said Form I of Amlodipine maleate is characterized by an X-ray powder diffraction pattern with 2θ values at about 4.459, 8.871, 10.593, 11.160, 11.488, 12.700, 13.296, 13.590, 15.694, 17.730, 18.156, 18.608, 19.208, 19.772, 20.126, 20.908, 21.523, 21.964, 22.985, 23.687, 24.584, 25.085, 25.538, 26.269, 26.753, 27.504, 28.095, 29.972, 30.433, 31.175, 31.824, 32.671, 33.564, 34.132, 36.223, 37.836, 38.776, 39.914.

40. A method of preventing hypertension comprising administering an effective amount of Form II of Amlodipine maleate to a patient in need thereof; wherein said Form II of Amlodipine maleate is characterized by an X-ray powder diffraction pattern with 20 values at about 4.421, 8.851, 10.147, 11.421, 12.032, 12.644, 13.071, 13.297, 13.490, 14.435, 14.838, 15.655, 17.052, 17.561, 18.072, 18.611, 19.186, 19.692, 20.078, 21.237, 21.958, 22.912, 23.536, 24.191, 24.493, 25.058, 25.932, 26.264, 26.717, 27.250, 28.051, 29.778, 31.728, 33.512, 33.987, 35.233, 37.085, 40.536.
